(19) United States
(12) Patent Application Publication
(10) Pub. No.: US 2018/0064895 A1
(43) Pub. Date: Mar. 8, 2018

(54) DEVICES, SYSTEMS AND METHODS FOR IMPROVED INTUBATION AND MANAGEMENT OF AIRWAYS

(71) Applicant: ALI SADOUGHI, NEW YORK, NY (US)

(72) Inventor: ALI SADOUGHI, NEW YORK, NY (US)

(21) Appl. No.: 15/698,118

(22) Filed: Sep. 7, 2017

Related U.S. Application Data
(63) Continuation-in-part of application No. 14/506,499, filed on Oct. 3, 2014, now abandoned.

(51) Int. Cl. A61M 16/04; A61B 1/005; A61B 1/018; A61B 1/267

(52) U.S. Cl. CPC A61M 16/0488; A61B 1/0051; A61B 1/267; A61B 1/018; A61M 16/0418

(57) ABSTRACT

Devices, systems and methods for improved placement of endotracheal tubes which have particular application for awake or sedated intubation, are described. An intubation scope having a handle, a flexible probe, and an articulatable tip where the handle has a control lever to cause and control articulation of the tip of the intubation scope. The system has a locking mechanism to hold the ET tube in desired position over and along the flexible probe during the intubation process. Specific methods of using this system for improvement of the intubation techniques are described.

### BACKGROUND OF THE INVENTION

### 1. Field of the Invention

The present invention relates to devices and methods for aiding in the intubation of patients for mechanical ventilation and management of the airways.

### 2. State of the Prior Art

Endotracheal intubation is a medical procedure that secures a patient's airway through the use of an endotracheal (ET) tube placed in the patient's trachea to facilitate gas exchange. Endotracheal intubation is routinely performed in operating rooms or in emergency situations mostly in intensive care Units or emergency rooms. Intubation is an invasive procedure often achieved with the use of a laryngoscope via direct visualization of the relevant physiological landmarks. However this method requires the patient to be anesthetized and sedated which in turn have negative clinical consequences, especially in severely sick patients.

In some circumstances characteristics of the patient may result in difficulties with intubation. Conditions that are associated with difficult endotracheal intubations include obesity, trauma (laryngeal fracture, mandibular or maxillary fracture, inhalation burn, cervical spine injury, temporomandibular joint dislocation), inadequate neck extension (rheumatoid arthritis, ankylosing spondylitiss, halo traction), anatomic variations (micrognathia, prognathism, large tongue, arched palate, short neck prominent upper incisors), presence of a foreign body in the upper airway, congenital anomalies (Pierre-Robin's syndrome, Treacher Collins' syndrome, laryngeal atresia, Goldenhar's syndrome, craniofacial dysostosis), infections (submandibular abscess, peritonsilar abscess, epiglottitis), tumors (cystic hygroma, hematoma), full stomach, or contraindications to sedative and anesthetic agents and muscle relaxants.

Patients having one or more of the characteristics associated with difficult intubations may be intubated with the help of endoscopic visualization of the airways. Endoscopic visualization is currently achieved with either a flexible bronchoscope or an endoscopic stylet.

The use of a flexible scope has the advantage of accommodating for awake intubation if needed. Flexible bronchoscopic intubation is known to be a safer method of intubation. However it is often not readily available and requires an operator with special training and experience. Because of these limitations, flexible bronchoscopic intubation, although safer, may be postponed or deferred and practically utilized as a rescue method when other methods have failed. Usually the encounter is during an emergency situation with a rapidly crashing patient.

Another shortcoming associated with flexible bronchoscopic intubation is delayed visualization of downstream airway including larynx during intubation. Additionally, there are delays associated with preparation time for flexible bronchoscopic intubation which can cause other disadvantages. Currently available flexible scopes are long and are difficult to use and manipulate. They are typically designed and constructed for use in bronchoscopy, not for endotracheal intubation. These shortcomings in the field can cause serious and life-threatening complications, especially in urgent situations when flexible bronchoscopic intubation is considered a rescue measure.

The other form of endoscopic visualization during intubation is the use of endoscopic stylets which are generally rigid. These are similar to the stylet that are usually used during conventional intubation. The stylet is a rigid or semi-rigid rod that can be formed in different shapes before use. They are used to stiffen the ET tube and help it pass via the upper airways and place it in the trachea. The endoscopic stylet has a camera or a lens at the tip which is connected to a proximal video monitor or an eye piece. We still notice problems with these kinds of rigid stylets with or without camera/lens at the tip. The rigid and semi-rigid stylets have their own problems due to lack of maneuverability to pass beyond tortuous and difficult airways. The other disadvantage of the semi-rigid and rigid stylets is the discomfort that they cause to patients. The patients need to be sedated which is not permitted in awake intubation, and is not safe in critically ill patients who are already suffering from other co-morbid conditions.

To address the shortcomings in the field, the present disclosure provides an airway management device referred to as AMD which can be used for difficult airways and also for awake intubation i.e. placement of a breathing tube into a patient's airway without the need for general anesthesia or other sedation. The AMD provides an easy, fast and secure method of intubation. The designed equipment is portable, affordable and easily accessible. Moreover the AMD operator requires only a short training session. The small size of the device makes it operator friendly and usable in a variety of clinical situations. One of the major applications of this invention is to provide a replacement for flexible bronchoscopes and also rigid and semi-rigid endoscopic stylets during intubation.

The small size, ease of use, and other characteristics of this device such as continuous oxygen supplementation during intubation, make it a desirable method of intubation during cardio-pulmonary resuscitation (CPR) which is one of the big challenges during this life-threatening situation. Using this device for securing the airway and intubating the patient is associated with a minimum interruption of cardiac compression.

Embodiments of this invention also provide a suction system to clear secretions and blood from upper airways and oral cavity, as well as a tool for removing foreign bodies from airways or other interventions on airways system, which is a complement to its function as an intubation device.

### SUMMARY OF THE INVENTION

As specified in the Background Section above, there is a need in the art to develop new therapeutic tools for securing and management of the airways, improving intubation, and particularly awake intubation.

Thus, in one embodiment of the invention the device comprises a handle and a probe. The probe further comprises an articulatable tip that includes a camera, a suction channel opening and a light, wherein the articulatable tip is controlled by a control mechanism. In the disclosed embodiments an endotracheal tube (ET tube) is removably disposed over the probe of the flexible scope.

In another embodiment the invention is a system for management of the airways and intubation comprises a flexible scope. The flexible scope further comprises a handle and a probe. The probe is shorter than the conventional scopes used in bronchoscopy with a length of about 30 to 45 cm. The probe further comprises an articulatable tip and the articulatable tip further comprises a camera, a suction channel opening, and a light. The articulatable tip is controlled by a forceful control mechanism via a connecting control lever on the handle to the tip. In some embodiments, an ET tube is removably disposed over the probe of the flexible scope. A locking device or mechanism holds the ET tube tight over the probe in a desired position. The controlled movement of the tip of the probe is able to simultaneously move the ET tube while it is loaded over the probe.

In another embodiment, the invention is a method of intubation comprising the steps of disposing an ET tube over the probe of a flexible scope and inserting the FT tube into position in a patient at the same time as the scope advances into the patient.

### BRIEF DESCRIPTION OF THE DRAWINGS

The above and other aspects, features and advantages of the present invention will be more apparent from the following description when taken in conjunction with the accompanying drawings, in which:

FIG. 1 shows an overall view of one embodiment of an airway management device (AMD) including a flexible scope in accordance with the present invention prior to mounting of an ET tube;

FIG. 1A is a schematic of an ET tube suitable for mounting on the scope shown in FIG. 1;

FIG. 1B is a schematic of the ET tube shown in FIG. 1A mounted on the scope shown in FIG. 1;

FIGS. 2A and 2B show an embodiment of the present invention illustrating the movement controls for maneuvering the articulatable tip of the flexible scope;

FIG. 3A shows a detailed longitudinal cross-sectional view of the functional components of the articulatable tip of the flexible scope;

FIG. 3B is an enlarged end view of the articulatable tip of the flexible scope;

FIG. 3C is a detailed longitudinal cross-sectional view of the functional components of the handle of the flexible scope shown in FIG. 1;

FIG. 4 is a schematic view of a modified embodiment of an AMD including a flexible intubating scope or probe having a uniform cross-section along its operative length;

FIG. 5 is an enlarged cross section of the probe taken along line 5-5 in FIG. 4;

FIG. 6 is a schematic representation of an ET tube similar to the one that is shown in FIG. 1a with the inflatable cuff expanded;

FIG. 7 is a schematic of a locking device or mechanism, in perspective, in the form of a plastic catheter tube clamp;

FIG. 8 illustrates an ET tube loaded over the intubating scope, with the tip of the probe retracted approximately 1 cm behind the distal end of the ET tube, and secured with a locking device, similar to the clamp shown in FIG. 7, over the probe; and

FIG. 9 is similar to FIG. 8 but shows the ET tube fixed by the locking device more proximally over the intubating scope probe, to expose the tip of the probe to project beyond the distal end of the ET tube.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context.

In one embodiment, the present invention (AMD) is a flexible scope loaded with an endotracheal tube (ET tube). The flexible scope further comprises a camera, a suction tip and a light at its tip. The scope used for the AMD is relatively short, sized to fit within an ET tube. The scope is sized so that it can fit different sizes of commercially available ET tubes. To accommodate the use of such tubes, the length of the flexible scope in some embodiments can range 30-45 cm with a presently preferred length of about 40 cm. It is intended that the device of the present invention will vary in size to maintain compatibility with commercially available ET tubes. Therefore, in practice the scope can be within a range of about 30 cm to 45 cm long. ET tube sizes may also have similar variance. A scope length of 40 cm will accommodate most conventional ET tubes. The external diameter of the probe should fit the internal diameter of different ET tubes. The smallest adult ET tube is size 6 (internal diameter of 6 mm). The external diameter of the probe portion of the scope should be of uniform diameter or cross-section but may be made in different diameter sizes to accommodate desired ET tubes (to fit adult and pediatric populations.) However, the probe may also be tapered or have a variable diameter.

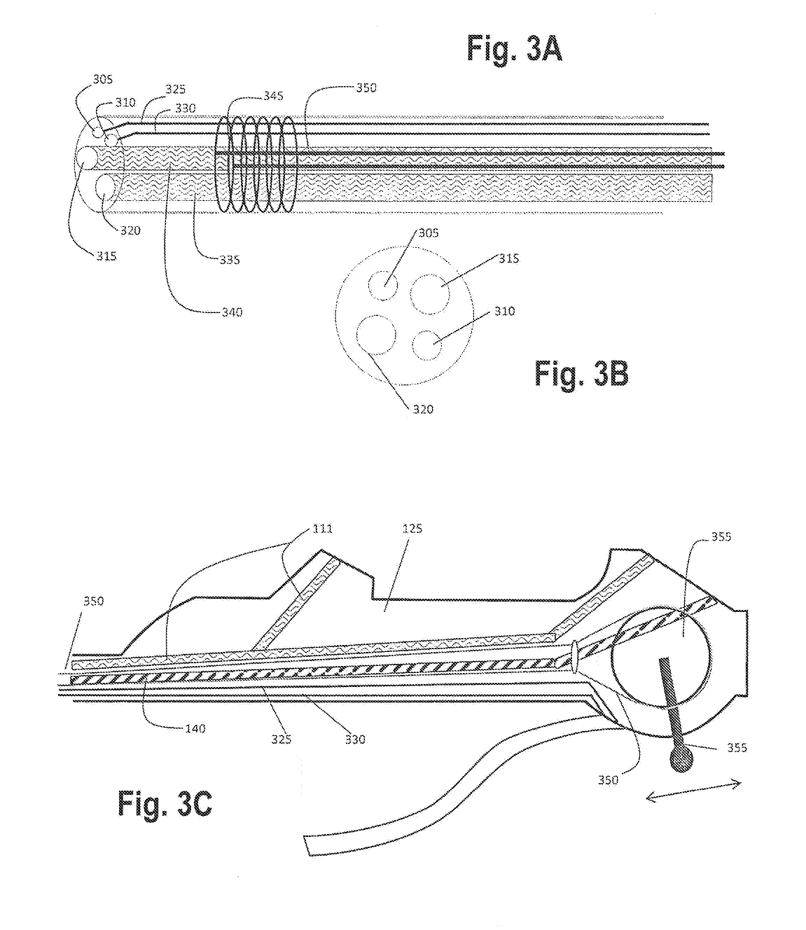

The ET tube is loaded onto the scope with the tip of the ET tube only slightly ahead of the tip of the flexible scope by a distance not greater than approximately 5 cm but preferably by about 1 cm. The tip of the scope can articulate in at least two directions. The articulation is controlled by a control mechanism on the handle of the scope (see FIG. 3C). Any known or conventional articulation mechanism may be used. Various appropriate control mechanisms for flexible scopes of this kind are readily known to those having skill in the art. The scope may be rotated by the operator so that the tip of the scope and the ET tube can be directed at any desired angle through the combination of rotation of the scope and articulation of the tip. The camera at the tip of the scope is operatively coupled to a video monitor which allows the operator to visualize physiological markers on the patient and thus guide the scope and the ET tube to the desired location. The scope may further comprise one or more channels which enables suctioning and clearance of secretions which might otherwise obscure visualization of relevant physiological markers. The suction capability may also be used to prevent aspiration of secretions into the lung and thereby protect the patient from aspiration pneumonitis which is associated with negative clinical outcomes. The channel(s) may also be used to administer pharmacological preparations such as anesthetizing agents, medications or supplemental oxygen. For example, the channel(s) may be used to apply local anesthetics while placing the ET tube, in order to increase patient comfort.

The flexible scope loaded with an ET tube is capable of airway intubation without the need for any other instrumentation placed in the mouth or upper airway of the patient. The camera at the tip of the scope provides visualization of the airway without the need for other bulky devices such as a laryngoscope or glidescope. The light provides illumination to aid in visualization. An optional channel in the flexible scope may be used for suction to clear the airway for better visualization and to remove blood, foreign bodies, regurgitated fluids, and the like, from the airway lumen. In order to keep the cost of production at a minimum and make for easy maintenance, the channel(s) in the flexible scope may be eliminated without affecting the operation of the present invention.

SHORT LENGTH OF SCOPE MAKE IT EASIER TO OPERATE. The flexible scope used in the AMD is shorter than conventional flexible bronchoscopes. The length can be within 30 to 45 cm which is only slightly longer than the largest ET tubes typically used. The shorter length of the scope used in the AMD makes it easier to be used at bedside and in emergency situations. Different scope sizes for adult and pediatric uses may be provided and each is designed for use with different size ET tubes. The shorter length of our probe compared to the conventional flexible bronchoscopes improves maneuvering of the distal end of the probe. The operator would have a much better control in hand movement to advance the tip of the probe using the handle and its control lever. This will allow the less experienced operator feel comfortable with intubation. One of the challenges of the flexible bronchoscopic intubation is that it needs an experienced operator who is proficient in use of the flexible scopes, which is eliminated with our invention.

THE SHORTER LENGTH OF THE SCOPE ELIMINATES THE NEED OF EXTRA ASSISTANCE DURING INTUBATION. The shorter length of the probe compared to the conventional flexible bronchoscopes allow the operator to free one hand which decrease the need for an assistance to perform other concurrent tasks during intubation. Using both hands is one of the obstacles of the current flexible bronchoscopic intubation.

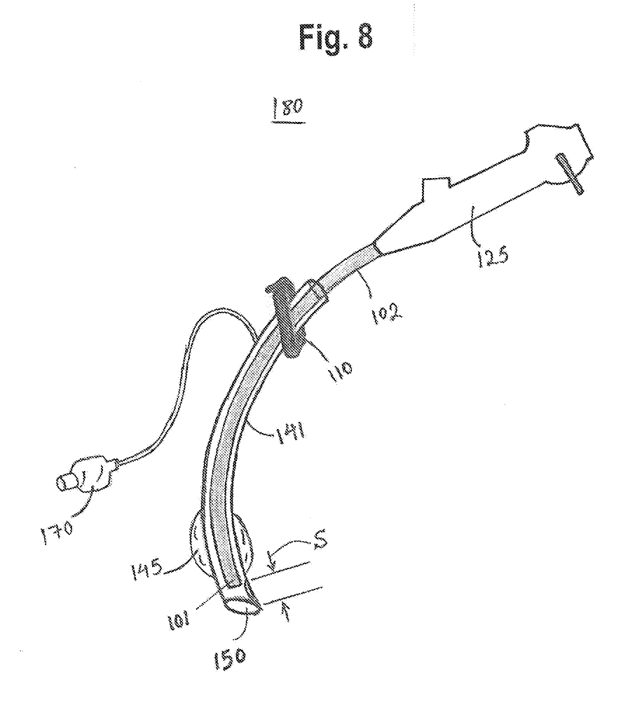

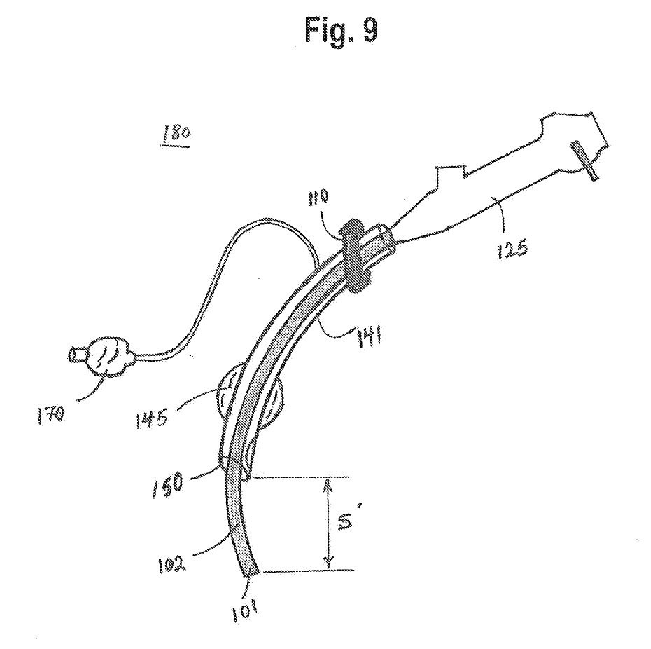

There is a locking mechanism which works as a clamp and will hold the ET tube against the probe in the desired position based on the size of the ET tube and the chosen method of intubation (FIG. 8 or 9). The locking mechanism may be any mechanism known to those having skill in the art, for example, a band, a clip or a clamp which can be tightened around the probe to hold the ET tube in the desired position over the probe. The locking mechanism prevents any movement of the ET tube over the probe while the intubation is being performed.

THE PROBE OF THIS INVENTION IS NOT RIGID LIKE ENDOSCOPIC STYLETS NOR IS FLIMSY LIKE FLEXIBLE BRONCHOSCOPES. The ET tube is splinted over the probe of the flexible scope. Splinting the flexible scope with an ET tube eliminates the need for a rigid bulky device such as a laryngoscope or glidescope. One of the obstacles of the flexible bronchoscopic intubations is that conventional bronchoscopes or endoscopic laryngoscopes are flimsy and not hard enough. The ET tube can be diverted into the esophagus during the time that ET tube is sliding over scope after the scope has passed the vocal cords. Also in circumstances with abnormalities in hypopharynx such as tumor or crowded airways, the conventional flexible scopes don't have enough strength to pass and find their way into trachea. By coupling of the ET tube and probe as in FIG. 8, the ET tube will splint the flexible scope and add to the rigidity of the probe enough to be able to overcome the resistance from the tissues in the hypopharynx. AMD's small flexible probe with the splinting support of the FT tube makes it an ideal device for any kind of airway intubation from awake to sedated patients and from emergent to elective cases.

The disadvantage of using rigid laryngoscope or any similar device such as Glidescope includes but not limited to: it is a bulky device—needed the use of extra hand—is uncomfortable for patient and requires the patient to receive more sedatives and anesthetics, so is not appropriate in awake intubation technique neither in patients who can't tolerate extra sedatives and anesthetics due to unstable medical condition—is a limiting factor by itself in patients with difficult airways and narrowed upper airways, small mouth opening, etc. In our claimed invention, as was explained in the original application, the need for using laryngoscope and glidescope is eliminated.

A lever, operatively coupled to the articulatable tip of the flexible scope allows an operator to articulate the tip of the scope. As the tip of the scope is articulated, it simultaneously articulates the ET tube that is splinted over the scope. In our invention, the designed tip of the probe is not only articulatable but also is forceful, meaning that by flexing the tip of the probe, the tip of the ET tube can be bent simultaneously as it is coupled in a position shown in FIG. 8 when the tip of the probe is about 1 cm behind the tip of the ET tube. It helps the ET tube to maneuver via difficult airways and reach to the trachea.

The intubation technique can be modified while using AMD, depending on different clinical scenarios that a provider may encounter during intubation. When the ET tube cannot be passed into trachea due to anatomical problem and severe narrowing of the upper airways, then the locking system will be released, push the probe inside the ET tube and let the tip of the probe pass the distal end of the ET tube. The probe can pass beyond the distal end of the ET tube up to about 10 cm when is coupled such as in FIG. 9. Then the lock will be tightened and the operator can manipulate the probe with hand movements over the handle and the control lever until it finds its way into the trachea. Then the ET tube will slide over the probe into trachea after the lock is released.

As shown in FIG. 8, the tip of the scope remains slightly inside and behind the tip of the ET tube so that the camera will not touch the airway wall while advancing the device into the airway and the vision won't be obscured. The visualization achieved by using AMD allows the operator to find the downstream airway and the larynx. As the tip of the ET tube stays ahead of the tip of the scope, the tip of the ET tube helps to push aside soft tissue and find the airway. The camera at the tip of the scope provides visualization without being blocked by oropharynx and hypopharynx wall, as shown in FIG. 8.

THIS INVENTION MAKE A FASTER VISUALIZATION OF THE LARYNX AND VOCAL CORDS. Using AMD the operator finds the downstream airway faster than conventional flexible bronchoscopic intubation or with endoscopic stylets; and advance the ET tube toward the trachea. The of ways of coupling of the ET tube with our probe will decrease the time of larynx visualization and shorten the time of intubation. (refer to FIG. 8 and 9) The scope may be rotated by the operator so that the tip of the scope and the ET tube can be directed at any desired angle through the combination of rotation of the scope and articulation of the tip.

AMD IS EASIER TO SET UP BEFORE USE COMPARED TO CONVENTIONAL BRONCHOSCOPES. The current available bronchoscopes are long and are difficult to manipulate especially in situations when the time is of essence. They are typically designed for bronchoscopy not for intubation. Additionally, there are usually delays due to their preparation time. Our invention is portable, easier to set up and use, and also more accessible.

Different types of ET tubes may be used with AMD. Some ET tubes are made of a softer material, while others are harder, being made from a harder plastic material. Depending on characteristics of the particular patient, such as the fullness of the retropharyngeal area, the width of the airway, the ease with which the scope is passed through the pharynx to find the larynx; operators may choose different ET tubes to better splint the flexible scope and maneuver it into the trachea. The harder, and more rigid the ET tube, the better the splint effect. On the other hand, softer, more flexible ET tubes are typically easier to use for awake intubation because of their increased maneuverability. The body of the ET tube and its tip are transparent in order to facilitate image capture by the camera at the tip of the flexible scope. While it is contemplated that the body of the ET tube is to be transparent, that is not a feature necessary for the operation of the invention.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure.

Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

It should be noted that the components in the figures are not necessarily to scale, emphasis instead being placed upon illustrating the principles of the invention. Moreover, in the figures, like reference numerals designate corresponding parts throughout the different views. However, like parts do not always have like reference numerals. Moreover, all illustrations are intended to convey concepts, where relative sizes, shapes and other detailed attributes may be illustrated schematically rather than literally or precisely.

In use, a video monitor is operatively coupled to the camera at the tip of the flexible scope. The monitor may be any video display known to those skilled in the art. The monitor may be made in various sizes. In some embodiments, the display may be attached to the flexible scope while in other embodiments, the display is located remotely and coupled to the camera by any connection known to those skilled in the art, such as, for example by a cord or wireless connection. The display may be operatively coupled to technology that would enable it to capture and save images and video clips of the procedure as it is performed.

Although the systems and devices disclosed herein have been designed for use in intubation (in awake or sedated patient), they may also be used for inspection of the pharynx, larynx and vocal cords. Additionally, the optional channel in the flexible scope may be used as a therapeutic device to advance different tools such as grab forceps, basket, cauterization devices, and other tools used with standard bronchoscopes for diagnostic and therapeutic purposes. Further, the AMD can be used for evaluation of swallowing and assessing the risk of aspiration.

The systems, devices and methods described herein offer a variety of advantages and benefits over the prior art including, but not limited to: (1) interruption of cardiac compression during cardio pulmonary resuscitation (“CPR”). The AMD provides an intubation technique that confers substantial benefits over the current intubation techniques typically used during CPR. By using a smaller device for intubating the patient and with the increased speed of intubation, it allows the personnel performing CPR to continue cardiac compressions with little or no interruption. In current practice using intubation devices of the prior art, chest compression has to be stopped for a substantial period of time when intubation of the patient is required. (2) Continuous oxygen supplementation during intubation. In current practice, effective oxygen supplementation is usually interrupted during the time it takes to intubate the patient. This can have detrimental and dangerous outcomes for the patient due to potentially severe hypoxemia. The AMD allows for the continued high flow of oxygen through channel(s) of the device in order to provide continuous oxygen delivery to the patients' upper airways. (3) High flow oxygen and ventilation is provided during resuscitation. As a result of the 2 previously enumerated benefits, using AMD during resuscitation and CPR will provide substantially improved oxygenation and ventilation during resuscitation. (4) Suctioning in order to clear secretions and blood from upper airways and oral cavity. As previously described above, the suction channel at the tip of the probe allows for the simultaneous suction of secretions and blood from the upper airways and oral cavity. This is beneficial both because the presence of secretions and blood can obscure airway visualization and make it difficult to intubate the airway, but also because the presence of secretions and blood can be hazardous to the patient if aspirated into the lungs.

This invention is able to help in this situation, because it causes no interruption of the cardiac compression while the patient is being intubated. It is a safe, secure and fast way of intubation for almost all kind of airways including difficult and unpredicted abnormalities in upper airways. Multiple mechanisms have been'known and published as a potential causal relationship between currently practiced tracheal intubation and poor outcomes during CPR. None of them are encountered with AMD for the following reasons:

First, tracheal intubation might lead to a prolonged interruption in chest compressions. Not expected with AMD.

Second, tracheal intubation could delay other interventions such as defibrillation or epinephrine administration. Not expected with AMD.

Third, delays in the time to successful intubation could result in inadequate ventilation or oxygenation by other means. Not expected with AMD.

Fourth, unrecognized esophageal intubation or dislodgement of the tube during the chest compression could lead to fatal outcomes. Not expected with AMD due to direct visualization of the downstream airways.

Potential beneficial effects of intubation during CPR include better control of ventilation and oxygenation as well as protection from aspiration. AMD is expected to help suction the aspirates from airways. Moreover, once an advanced airway is established, chest compressions may be provided in a more continuous fashion. With AMD, chest compression is not needed to be interrupted at all.

### Referring Now to the Figures:

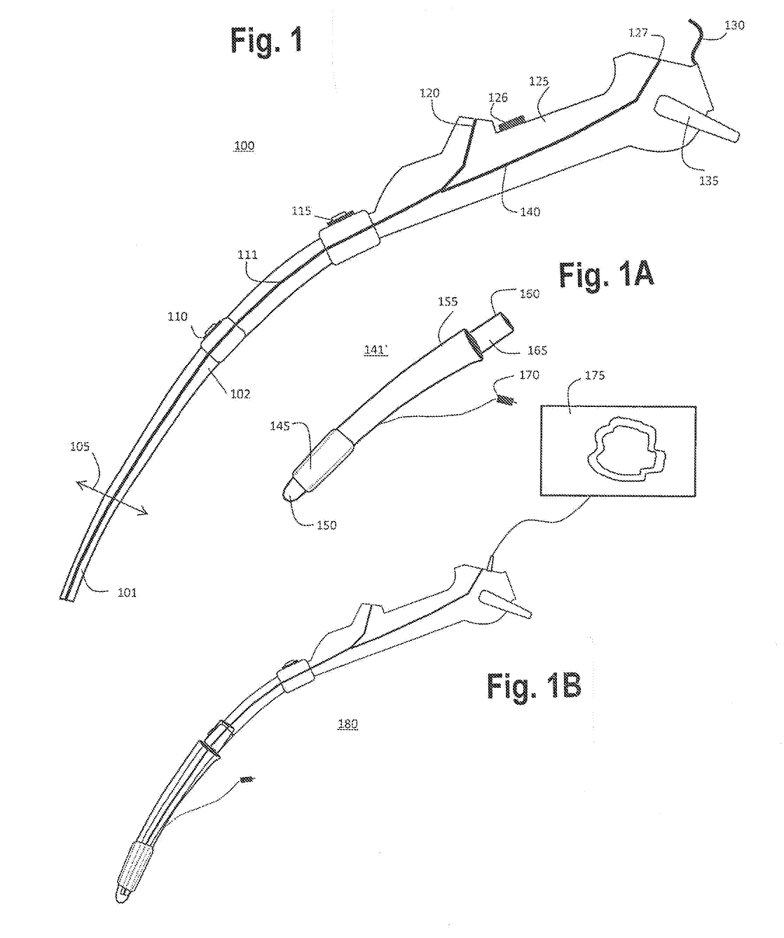

FIG. 1, 1A and 1B show an overall view of an embodiment of the present invention illustrating various parts of the invention. In one preferred embodiment a flexible scope 100 comprises a handle 125 and a flexible probe 102. The probe 102 further comprises an articulatable tip 101 that can articulate in directions 105 in response to operator manipulation of a tip control mechanism, such as, for example, a control lever 135 on the handle 125 of the scope. The articulatable tip, in some embodiments, further comprises various components or features such as lights, cameras, channel openings for suction/pharmaceutical preparation delivery, and the like. The various functions of the tip components are controlled via a control interface 126 on the handle of the scope. The probe 102 is adapted to fit inside an ET tube (141) (FIG. 1A). A suitable lock or clamp can be used to longitudinally secure the ET tube in place by engaging the ET tube with a locking mechanism 110. In some embodiments, the scope further comprises a channel or a plurality of channels (111 and 140) that run the length of the probe 102 and handle 125 respectively. A device with a plurality of channels, such as 2 channels, can provide substantial benefits. For example, one channel can be used for oxygen supplementation while the other one is used for suction at the same time. The second channel may alternatively be used to pass different endoscopic tools that are routinely used during bronchoscopy or laryngoscopy (including, but not limited to, biopsy, electro-cauterization, cryo-ablation, foreign body retrieval basket tools, and the like). A device with a single channel provides some of the advantages of the multiple channel device but is less expensive to manufacture, maintain and clean. A device without channels would be the least expensive to maintain and manufacture. The channels) communicate with connections 120, 127 on the handle 125 to link the scope with suction/agent delivery apparatus. In some embodiments the scope may be operatively coupled, such as by a wire 130, to a video display 175 to provide visual information to the operator. The scope is designed to operate with ET tubes 141. ET tubes used in the present invention comprise a body 155, a tip 150, an inflatable cuff 145, a connection section 165, a connector 160 for attachment to a ventilator, and a balloon connector or coupling portion 170 operatively coupled to the cuff to monitor the pressure inside the cuff while the cuff is inflated. The ET tube 141 fits over the probe portion of the scope 180.

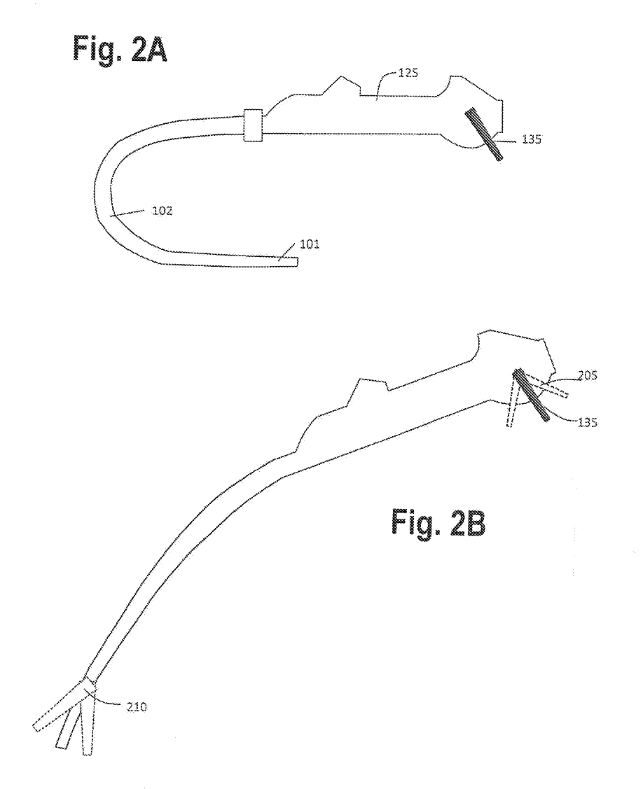

FIGS. 2A and 2B show an embodiment of the invention illustrating the movement of the articulatable tip 101 in response to operator control. The scope 100 of the present invention comprises a handle 125 and a control lever 135 on the handle and the probe portion 102. The probe portion 102 is flexible to facilitate use in patients. The control lever 135 is operatively connected to the articulatable tip 101. In response to operator manipulations of the lever (e.g. positions 205) the articulatable tip 101 articulates in at least 2 directions 210.

FIG. 3A shows a detailed view of the various components or features present in some embodiments of the articulatable tip 101 and FIG. 3C shows detailed views of the components and control mechanisms in the handle 125. The articulatable tip 101, in some embodiments comprises various functional components such as channel openings 315, 320, a camera 305, and a light 310. Wired connections 330, 325 operatively connect the camera and/or light to the control interface on the handle of the scope. Channel openings 315, 320 are in communication with channels 335, 340 in order to provide suction and material delivery at the articulatable tip. In order to facilitate articulation of the articulatable tip, support structures, such as metal rings 345 are fixed to an inside surface of the articulatable tip. Control wires 350 are attached to the metal rings 345 and to a rotary control wheel 355. In use, when the operator manipulates the control lever 355 (FIG. 3C) the lever turns the rotary control wheel 355 which in turn applies force to the control wires 350 which alternatively push and pull on the metal rings 345 attached to the articulatable tip which cause articulation of the articulatable tip. FIG. 3B shows an end view of the articulatable tip 101 showing the camera 305, light 310 and channel openings 315, 320.

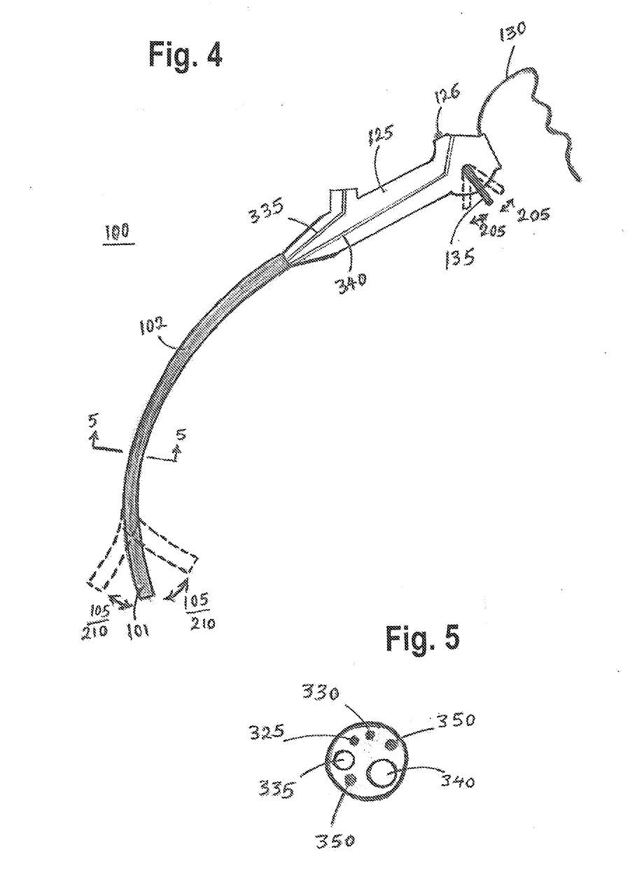

Referring to FIG. 4-6, an alternate embodiment of the probe 102 of the flexible scope 100 has a substantially uniform diameter or cross-section along its operative length. FIG. 5 illustrates the cross section of the flexible probe taken along line 5-5 in FIG. 4 and shows the elements passing via the probe including the channels 335 and 340, the wires 325 and 330 connecting the light and camera to the control interface on the handle of the scope and control wires 350 that connect the articulable tip to the control level on the handle. As indicated, the probe may omit channels or conduits (335, 340) to decrease the cost of manufacturing and maintenance without eliminating the main function of the device.

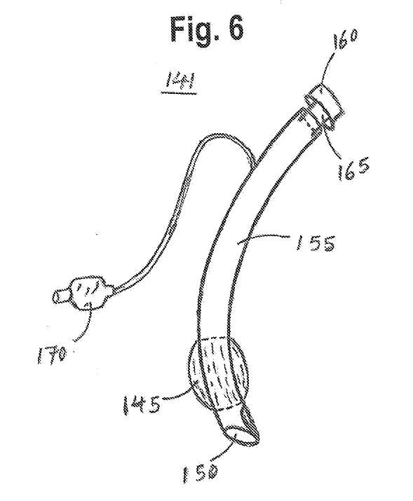

FIG. 6 is similar to the inset in FIG. 1 and shows details of the ET tube 141, with the balloon portion or inflatable cuff 145 expanded.

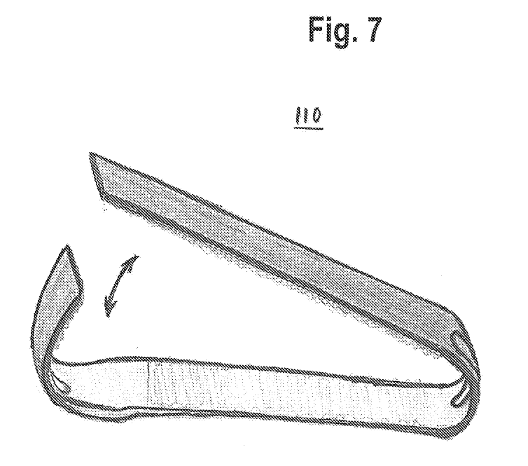

FIG. 7 illustrates one embodiment of a locking device that can be used to secure the ET tube 141 to the probe 102. The locking device 110 may be any suitable device that secures the ET tube onto the probe to prevent relative longitudinal movements of the ET tube along the probe once locked in place. For example, any known clamp designs can be used that can apply inward forces or pressure on the ET tube. Circumferential or diametrically opposing forces can be used. Example of such clamps include plastic catheter tube clamp.

In FIG. 7, the device 110 is similar to a catheter tube clamp that can adjust the diagonal forces or pressures on an item extending through the clamp. In FIG. 7 the plastic catheter tube clamp is an adjustable clamp with serrated jaw which apply pinching force on ET tube and hold it against the probe. It springs open when released and can be operated with one hand.

FIG. 8 illustrates the ET tube 141 secured on the probe 102 with the tightened clamp 110, so that the articulatable tip 101 is retracted within the ET tube 141 and does not extend distally through or beyond the distal end 150 of the ET tube. As indicated, the spacing “s” is advantageously approximately 1 cm although this distance is not critical.

In FIG. 9 the ET tube 141 is secured in place at a more proximate position on the probe 102 with the tightened clamp 110, such that the probe tip 101 projects or extends a distance “S′” within the range of 0-10 cm beyond the distal open end 150 of the ET tube 141. It is evident to those skilled in the art that with such construction the flexible scope can be used in a number of different modes as described and accommodate different lengths ET tubes.

In some embodiments the intubation scope will be sized for adults. In other embodiments, the intubation scope will be sized for a pediatric population.

While the invention has been shown and described with reference to certain embodiments thereof, it will be understood by those skilled in the art that various changes in form and detail may be made therein without departing from the spirit and scope of the invention as defined by the appended claims and their equivalents.

What is claimed:

1. An intubation system comprising:
a flexible scope comprising a handle; an elongate flexible probe having a length within the range of 30-45 cm and defining a longitudinal direction connected to said handle at one end and having an articulatable tip at an opposing end;
control means in proximity to said handle operatively coupled to said articulatable tip such that said articulatable tip can articulate in response to manipulations of said control means;
an elongate endotracheal tube (ET tube) having a length smaller than the length of said flexible probe, said ET tube having proximal and distal ends and provided with an elongate internal channel dimensioned to releasably receive at least a portion of said opposing end of said flexible probe including said articulatable tip;
said articulatable tip being sufficiently forceful to be able to simultaneously bend the tip of the loaded ET tube while is controlled by manipulations of said control means; and
locking means for selectively locking said ET tube along a plurality of selected longitudinal positions along said opposing end of said flexible probe to manipulate or guide at least said distal end of said ET tube by said articulatable tip while selectively positioning said opposing end of said flexible probe either in a retracted position within or a projected position beyond said distal end, whereby different length ET tubes can be manipulated by said flexible probe.

2. An intubation system as defined in claim 1, wherein said flexible probe is more flexible than said ET tube.

3. An intubation system as defined in claim 1, wherein said flexible probe includes the following: optional one or two longitudinal channel or passaway, a wire for a camera, a wire for a light and control wires for manipulating said articulatable tip.

4. An intubation system as defined in claim 1, wherein said locking means comprises an adjustable plastic catheter tube clamp.

5. An intubation system as defined in claim 4, wherein said plastic catheter tube clamp is a one-hand operatable adjustable clamp with a serrated jaw to apply a pinching force on said ET tube and hold it against the probe until manually released.

6. An intubation system as defined in claim 1, wherein said locking means comprises a catheter clamp for applying diametrically inward compression forces on said ET tube.

7. An intubation system as defined in claim 2, wherein said elongate flexible probe and said ET tube are movably adjustable relative to each other along a direction corresponding to said ET tube elongate internal channel to allow selective positioning of said flexible probe opposing end relative to said ET tube distal end.

8. An intubation system as defined in claim 7, wherein said ET tube distal end is movable between a retracted position over and upstream of said opposing end of said flexible probe and an extended position downstream of said opposing end distally beyond or ahead of said flexible probe.

9. An intubation system as defined in claim 8, wherein said ET tube distal end is positionable to extend approximately 0-5 cm beyond said opposing end of said probe in said extended position.

10. An intubation system as defined in claim 1, wherein said articulated tip can be articulated in at least two directions.

11. An intubation system as defined in claim 1, wherein said opposing end of said probe is positionable to extend a distance of 0-10 cm beyond said ET tube distal end.

12. An intubation system as defined in claim 1, wherein said ET tube has a predetermined flexibility and said control means and said articulatable tip can generate deflecting or bending forces on said distal end of said ET tube to deflect or bend said distal end when coupled to overcome said predetermined flexibility, wherein said articulated tip is forceful enough to be able to simultaneously bend the tip of the loaded ET tube while is controlled by manipulations of said control means.

13. An intubation system as defined in claim 1, wherein said locking means comprises a clamp that can deform and compress said FT tube around said flexible probe to hold said ET tube in a desired position relative to said flexible probe.

14. An intubation system as defined in claim 1, wherein said flexible probe has a length of approximately 40 cm.

15. An intubation system as defined in claim 1, wherein said probe has an outer diameter of approximately 5.5 cm.

16. An intubation system as defined in claim 1, wherein said probe is less firm than an endoscope stylet or conventional intubating stylets.

17. An intubation system comprising:
a flexible scope comprising a handle; an elongate flexible probe having a predetermined length and defining a longitudinal direction connected to said handle at one end and having an articulatable tip at an opposing end; and
control means in proximity to said handle operatively coupled to said articulatable tip such that said articulatable tip can articulate in response to manipulations of said control means; an elongate ET tube (ET tube) having length smaller than the length of said flexible probe, said ET tube having proximal and distal ends and provided with an elongate internal channel dimensioned to releasably receive at least a portion of said opposing end of said flexible probe including said articulatable tip;
locking means for selectively locking said ET tube along a plurality of selected longitudinal positions along said opposing end of said flexible probe to manipulate or guide at least said distal end of said ET tube by said articulatable tip while selectively positioning said opposing end of said flexible probe either in a retracted position within or a projected position beyond said distal end, whereby different length ET tubes can be manipulated by said flexible probe.

18. A method of intubating a patient comprising the steps of coupling a flexible probe having a length between 30-45 cm and defining a longitudinal direction and connected to a handle at one end and having an articulatable tip at an opposing end with an endotracheal tube (ET tube) having a length smaller than a length of the flexible probe;
selectively positioning the ET tube to place the free end of the probe in a retracted position within or in an extended position in relation to the end or tip of the ET tube;
securing the ET tube in the selected position to prevent relative longitudinal movements between the probe and the ET tube;
advancing the probe while loaded with the ET tube into the patient's airway to locate the patient's downstream airway; and
maneuvering the free end of the probe with a remote actuator on the handle through said probe to guide the ET tube past the patient's vocal cords into the windpipe to position the ET tube inside the trachea for intubation.

19. A method of intubating a patient as defined in claim 18, wherein the ET tube is arranged to position the free end of the probe in a retracted position within the tip of the ET tube a distance of 1-5 cm.

20. A method of intubating a patient as defined in claim 18, wherein the ET tube is arranged to position the free end of the probe in an extended position beyond the tip of the ET tube a distance of 0-10 cm.
